(19) United States
(12) Patent Application Publication
(10) Pub. No.: US 2022/0401462 A1
(43) Pub. Date: Dec. 22, 2022

(54) COMBINATION PREPARATIONS OF 3-N-FORMYLHYDROXYLAMINOPROPYL PHOSPHONIC ACID DERIVATIVES OR 3-N-ACETYLHYDROXYLAMINOPROPYL PHOSPHONIC ACID DERIVATIVES WITH CLINDAMYCIN AND ARTESUNATE

(71) Applicant: DMG Deutsche Malaria GmbH, Hamburg (DE)

(72) Inventors: David HUTCHINSON, Westerham (GB); Björn Friedrich LINDEMANN, Hamburg (DE)

(21) Appl. No.: 17/769,875

(22) PCT Filed: Oct. 19, 2020
(86) PCT No.: PCT/EP2020/079401

§ 371 Date: Apr. 18, 2022

(30) Foreign Application Priority Data
Oct. 19, 2019 (EP) 19204213.3

(51) Int. Cl. A61K 31/662; A61K 31/7052; A61K 31/357

(52) U.S. Cl. CPC A61K 31/662; A61K 31/7052; A61K 31/357

(57) ABSTRACT

The present invention relates to pharmaceutical preparations containing as active ingredients 3-N-formylhydroxylaminopropylphosphonic acid derivatives or 3-N-acetylhydroxylaminopropylphosphonic acid derivatives in combination with clindamycin and artesunate for the treatment of malaria, in particular for the acute therapy of cerebral malaria, and to an associated dosage, in particular for the patient group of children.

The present invention relates to pharmaceutical preparations containing as active ingredients 3-N-formylhydroxylaminopropylphosphonic acid derivatives or 3-N-acetylhydroxylaminopropylphosphonic acid derivatives in combination with clindamycin and artesunate for the treatment of malaria, in particular for the acute therapy of cerebral malaria, and to an associated dosage, in particular for the patient group of children.

The use of 3-N-formylhydroxylaminopropylphosphonic acid derivatives and 3-N-acetylhydroxylaminopropylphosphonic acid derivatives for the prophylactic and therapeutic treatment of infectious processes, in particular infections caused by unicellular parasites (also called protozoa for the purposes of this invention) or multicellular parasites, is already known from DE 198 25 585 A1. A bacterial efficacy is already described in DE 27 33 658 A1. Even though these compounds show good results in the treatment of infections caused by parasites or bacteria, these drugs also show undesirable side effects.

WO2002000208A2 describes pharmaceutical preparations containing as active ingredients 3-N-formylhydroxylaminopropylphosphonic acid derivatives or 3-N-acetylhydroxylaminopropylphosphonic acid derivatives in combination with clindamycin or artesunate for the treatment of malaria.

The present invention has therefore set itself the task of enhancing the effect of these pharmaceutical agents by means of further therapeutic instructions.

The aim here is to increase the therapeutic range of application of these pharmaceutical agents, especially for the treatment of problematic groups such as children with cerebral malaria.

The aim is to enhance the antiparasitic effect so that these pharmaceutical agents can be administered in appropriate and optimized doses and thus achieve rapid therapeutic success in cerebral malaria.

However, such combination preparations behave very differently in terms of stability, efficacy, pharmacology, etc., especially depending on the course of the disease in a malaria illness and its symptoms (fever, etc.) as well as course forms.

Surprisingly, it has now been found that

3-N-formylhydroxylaminopropylphosphonic acid derivatives and/or 3-N-acetylhydroxylaminopropylphosphonic acid derivatives in combination with clindamycin and artesunate—hereinafter referred to as “combination preparation” or “composition”—is particularly suitable for the treatment of cerebral malaria.

In the context of this invention, “cerebral malaria” is understood to be a form of progression or complication of a malaria infection affecting the brain, in which an accompanying loss of consciousness can occur up to coma of the patient, and in particular such patients can exhibit comatose phases which usually last longer than half an hour and are frequently accompanied by convulsive twitching. Other symptoms may include abnormal movements of the eyes, spasmodically closed mouth with simultaneous rubbing of the rows of teeth against each other or pouting, slight stiffness of the neck, and a whole range of movement disorders.

Cerebral malaria causes death in about 20% of adults and 15% of children. About 10% of surviving children and 2% of adults, most of whom had other symptoms of severe malaria, suffer permanent brain damage.

Clindamycin or methyl-6-amino-7-chloro-6,7,8-trideoxy-N-[(2S,4R)-1-methyl-4-propylprolyl]-1-thio-β-L-threo-D-galactooctopyranoside has the formula (a):

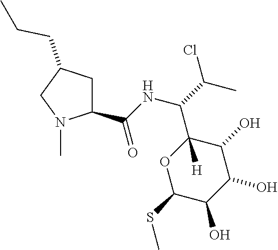

Artesunate or (3R,5aS,6R,8aS,9R,10S,12R,12aR)-decahydro-3,6,9-trimethyl-3,12-epoxy-12H-pyrano[4,3-j]-1,2-benzodioxepin-10-ol hydrogen succinate has the formula (b):

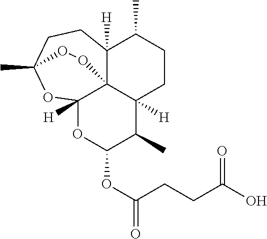 [SMILES: C[C@H]1[C@H](OC(=O)CCC(=O)O)O[C@@H]2O[C@]3(C)CC[C@H]4[C@H](C)CC[C@@H]1[C@@]24OO3]

3-N-formyl-hydroxylamino-propylphosphonic acid derivatives and 3-N-acetyl-hydroxylamino-propylphosphonic acid derivatives are compounds of formula (I) according to the invention.

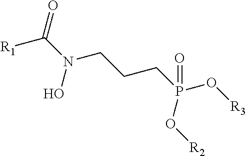

wherein R₁ is selected from the group consisting of hydrogen and methyl, and wherein R₂ and R₃ are independently selected from the group consisting of hydrogen, substituted or unsubstituted alkyl, substituted or unsubstituted acyl, substituted or unsubstituted aryl, substituted or unsubstituted aralkyl, substituted or unsubstituted cycloalkyl, a substituted or unsubstituted silyl, substituted or unsubstituted heterocyclic radical, or together form a substituted or unsubstituted C₁₋₅ alkyl chain, wherein the alkyl groups may be saturated or contain one or more double bonds or triple bonds.

According to the invention, this combination preparation also includes the respective salts or any stereoisomers.

It is particularly preferred that R₁, R₂ and R₃ is hydrogen or formula I is fosmidomycin, including salts and stereoisomers thereof.

Surprisingly, the combination preparation according to the invention can bring about a 100% clearance of the malaria pathogen Plasmodium falciparum within 3 days in a group of patients and, in particular, a decrease in malaria-induced fever to normal temperature within 48 hours, especially in the case of simultaneous application. Such an impressive effect could not have been predicted. 100% parasite clearance and normal body temperature means that patients are parasite and symptom free.

Particularly advantageously, clindamycin and formula I, preferably fosmidomycin prevent a delay in action in the case of wholly or partially artesunate-resistant malaria pathogens (Plasmodium falciparum), so that even such patients can be treated who have been infected with artesunate-resistant Plasmodium falciparum.

In a preferred embodiment, the invention therefore relates to a combination preparation according to the invention as well as to a medicament for use in the treatment of cerebral malaria.

Furthermore, it is advantageous that the combination preparation according to the invention allows treatment also in the presence of further bacterial infections, which, for example, are also introduced via the malaria pathogen or are not repelled as a result of the malaria disease, since clindamycin and formula I, preferably fosmidomycin, have a broad anti-bacterial effect.

In another preferred embodiment, the patient is selected from the group of children 0-14 years, in particular 0-10 years.

In another preferred embodiment, the dose for use in the treatment of cerebral malaria is three times the amount (weight in mg) of an active ingredient of formula I, in particular fosmidomycin versus clindamycin and/or 7.5 times-to fifteen times the amount (weight in mg) of an active ingredient of formula I, in particular fosmidomycin versus artesunate.

In another preferred embodiment, the dose for use in the treatment of cerebral malaria is 1-6 mg of artesunate, 5-15 mg of clindamycin and 10-40 mg according to formula I, in particular fosmidomycin per kg of body weight of a patient, preferably a dose of 2-4 mg of artesunate, 10 mg of clindamycin and 30 mg according to formula I, in particular fosmidomycin per kg of body weight of a patient.

For example, a dose for a child of 25 kg body weight is 25-150 mg of artesunate, 125-375 mg of clindamycin and 250-1,000 mg according to formula I, in particular fosmidomycin per kg of body weight of a patient, preferably a dose of 50-100 mg of artesunate, 250 mg of clindamycin and 750 mg according to formula I, in particular fosmidomycin.

Furthermore, it is preferred that these doses according to the invention be used in acute therapy for the treatment of cerebral malaria.

In another preferred embodiment, these doses are administered within 12 hours.

Furthermore, it is preferred that these doses be administered consecutively every 12 hours for 3-5 days.

Special features of the above definitions of formula I and suitable examples thereof are given below:

“Acyl” is a substituent derived from an acid such as an organic carboxylic acid, carbonic acid, carbamic acid or the thioacid or imidic acid corresponding to each of the above acids, or from an organic sulfonic acid, each of these acids comprising aliphatic, aromatic and/or heterocyclic groups in the molecule, and carbamoyl or carbamimidoyl.

Aliphatic acyl groups are acyl radicals derived from an aliphatic acid, which include the following:

Alkanoyl (e.g. formyl, acetyl, propionyl, butyryl, isobutyryl, valeryl, isovaleryl, pivaloyl etc.); alkenoyl (e.g. acryloyl, methacryloyl, crotonoyl etc.).); alkylthioalkanoyl (e.g. methylthioacetyl, ethylthioacetyl, etc.); alkanesulfonyl (e.g. mesyl, ethanesulfonyl, propanesulfonyl, etc.); alkoxycarbonyl (e.g. Methoxycarbonyl, Ethoxycarbonyl, Propoxycarbonyl, Isopropoxycarbonyl, Butoxycarbonyl, Isobutoxycarbonyl etc.); Alkylcarbamoyl (e.g. Methylcarbamoyl etc.); (N-alkyl)-thiocarbamoyl (e.g. (N-methyl)-thiocarbamoyl etc.); alkylcarbamimidoyl (e.g. methylcarbamimidoyl etc.); oxalo; alkoxalyl (e.g. methoxalyl, ethoxalyl, propoxalyl etc.).

In the above examples of aliphatic acyl groups, the aliphatic hydrocarbon moiety, in particular the alkyl group or alkane moiety, may optionally have one or more suitable substituents, such as amino, halogen (e.g. fluorine, chlorine, bromine, etc.), hydroxy, hydroxyimino, carboxy, alkoxy (e.g. methoxy, ethoxy, propoxy, etc.), alkoxycarbonyl, acylamino (e.g. benzyloxycarbonylamino, etc.), acyloxy (e.g. acetoxy, benzoyloxy, etc.) and the like.), alkoxycarbonyl, acylamino (e.g. benzyloxycarbonylamino, etc.), acyloxy (e.g. acetoxy, benzoyloxy, etc.) and the like; preferred aliphatic acyl radicals having such substituents include alkanoyls substituted with amino, carboxy, amino and carboxy, halogen, acylamino or the like.

Aromatic acyl radicals are those acyl radicals derived from an acid having a substituted or unsubstituted aryl group, wherein the aryl group may include phenyl, toluyl, xylyl, naphthyl and the like; suitable examples are given below: Aroyl (e.g. benzoyl, toluoyl, xyloyl, naphthoyl, phthaloyl, etc.); aralkanoyl (e.g. phenylacetyl, etc.); aralkenoyl (e.g. cinnamoyl, etc.).); aryloxyalkanoyl (e.g. phenoxyacetyl, etc.); arylthioalkanoyl (e.g. phenylthioacetyl, etc.); arylaminoalkanoyl (e.g. N-phenylglycyl, etc.); arenesulfonyl (e.g. E.g. benzenesulfonyl, tosyl or toluenesulfonyl, naphthalenesulfonyl, etc.); aryloxycarbonyl (e.g. phenoxycarbonyl, naphthyl-oxycarbonyl, etc.); aralkoxycarbonyl (e.g. N-phenylglycyl, etc.).); aralkoxycarbonyl (e.g. benzyloxycarbonyl etc.); arylcarbamoyl (e.g. phenylcarbamoyl, naphthylcarbamoyl etc.); arylglyoxyloyl (e.g. phenylglyoxyloyl etc.).

In the above examples of aromatic acyl groups, the aromatic hydrocarbon moiety and/or the aliphatic hydrocarbon moiety (in particular, the alkane moiety) may optionally have one or more suitable substituents, such as those already indicated as suitable substituents for the alkyl group or alkane moiety. As an example of preferred aromatic acyl moieties having particular substituents, aroyl substituted with halogen and hydroxy or with halogen and acyloxy and aralkanoyl substituted with hydroxy, hydroxyimino, dihaloalkanoyloxyimino are understood, as well as arylthiocarbamoyl (e.g. phenylthiocarbamoyl etc.); arylcarbamimidoyl (e.g. phenylcarbamimidoyl etc.).

A heterocyclic acyl radical is understood to be an acyl radical derived from an acid with a heterocyclic group, such as follows:

A heterocyclic carbonyl in which the heterocyclic radical is an aromatic or aliphatic 5- to 6-membered heterocycle having at least one heteroatom selected from the group consisting of nitrogen, oxygen and sulfur (e.g., thiophenyl, furoyl, pyrrolecarbonyl, nicotinoyl, etc.);

Heterocyclic alkanoyl in which the heterocyclic moiety is 5- to 6-membered and has at least one heteroatom selected from the group consisting of nitrogen, oxygen and sulfur (e.g., thiophenyl acetyl, furylacetyl, imidazolyl propionyl, tetrazolylacetyl, 2-(2-amino-4-thiazolyl)-2-methoxyiminoacetyl, etc.) and the like.

In the above examples of heterocyclic acyl radicals, the heterocycle and/or aliphatic hydrocarbon moiety may optionally have one or more suitable substituents, such as the same ones indicated as suitable for alkyl and alkane groups.

“Alkyl group” or “alkyl”, unless otherwise defined, is a straight or branched chain alkyl radical containing up to 26 carbon atoms, but preferably C1-C6 with methyl, ethyl, propyl, isopropyl, butyl, isobutyl, tert-butyl, pentyl, hexyl and structural isomers thereof. It can be substituted, for example, with hydroxy, amino, halogen (e.g. fluorine, bromine, chlorine), oxo radicals and alkoxy radicals, such as methoxy, ethoxy radicals.

“Cycloalkyl” preferably stands for an optionally substituted C₃₋₈ cycloalkyl; suitable possible substituents include alkoxy (e.g. methoxy, ethoxy, etc.), halogen (e.g. fluorine, chlorine, bromine, etc.), nitro and the like.

“Aryl” means an aromatic hydrocarbon radical, such as phenyl, naphthyl, etc., which may optionally have one or more suitable substituents such as alkyl, alkoxy (e.g. methoxy, ethoxy, etc.), trifluoromethylene, halogen (e.g. fluorine, chlorine, bromine, etc.), nitro and the like.

“Aralkyl” includes mono-, di-, triphenyl alkyls such as benzyl, phenethyl, benzhydryl, trityl and the like, wherein the aromatic moiety may optionally have one or more suitable substituents such as alkoxy (e.g. methoxy, ethoxy, etc.), halogen (e.g. fluorine, chlorine, bromine, etc.), nitro and the like.

When using combination therapy, it is possible to administer the active ingredients in a so-called fixed combination, i.e. in a single pharmaceutical formulation containing all three active ingredients, or to choose a so-called free combination, in which the active ingredients can be applied in the form of separate pharmaceutical formulations simultaneously or simultaneously but also one after the other—time-delayed.

The combination preparation according to the invention is effective against bacteria and unicellular and multicellular parasites, especially in the treatment and prevention against pathogens of malaria (Plasmodium).

All the indications mentioned, together with the associated symptoms, are described in the Pschyrembel®, de Gruyter, Berlin.

The pharmaceutically active agents can be prepared in the form of pharmaceutical preparations in dosage units. This means that the preparation is in the form of individual parts, e.g. tablets, dragees, capsules, pills, suppositories and ampoules, the active ingredient content of which corresponds to a fraction or a multiple of a single dose. The dosage units may contain, for example, 1, 2, 3 or 4 single doses or ½, ⅓ or ¼ of a single dose. A unit dose preferably contains the amount of active ingredient administered in one application, which usually corresponds to a whole dose, a half dose or a third or a quarter of a daily dose.

Non-toxic, inert pharmaceutically acceptable excipients are understood to be solid, semi-solid or liquid diluents, fillers and formulation aids of any kind.

Preferred pharmaceutical preparations are tablets, coated tablets, capsules, pills, granules, suppositories, solutions, suspensions and emulsions, pastes, ointments, gels, creams, lotions, powders and sprays. Tablets, coated tablets, capsules, pills and granules may contain the active ingredient(s) in addition to the usual excipients, such as a) fillers and extenders, e.g., starches, lactose, cane sugar, glucose, mannitol and silica, b) binders, e.g., carboxymethylcellulose, alginates, gelatin, polyvinylpyrrolidone, c) humectants, e.g., glycerol, d) disintegrants, e.g. agar-agar, calcium carbonate and sodium carbonate, e) dissolution retarders, e.g. kerosene, and f) resorption accelerators, e.g. quaternary ammonium compounds, g) wetting agents, e.g. cetyl alcohol, glycerol monostearate, h) adsorbents, e.g. kaolin and bentonite, and i) lubricants, e.g. talc, calcium and magnesium stearate and solid polyethylene glycols or mixtures of the substances listed under a) to i).

The tablets, coated tablets, capsules, pills and granules may be provided with the usual coatings and shells, optionally containing opacifying agents, and may also be formulated to deliver the active ingredient(s) only or preferentially in a specific part of the intestinal tract, optionally in a delayed manner, wherein polymer substances and waxes, for example, may be used as embedding materials.

The active ingredient(s) may also be present in microencapsulated form, optionally with one or more of the excipients indicated above.

Suppositories may contain, in addition to the active ingredient(s), the usual water-soluble or water-insoluble excipients, e.g., polyethylene glycols, fats, e.g., cocoa fat, and higher esters (e.g., C14 alcohol with C16 fatty acid) or mixtures of these substances.

Ointments, pastes, creams and gels may contain, in addition to the active ingredient(s), the usual excipients, e.g. animal and vegetable fats, waxes, kerosenes, starch, tragacanth cellulose derivatives, polyethylene glycols, silicones, bentonites, silica, talc and zinc oxide or mixtures of these substances.

Powders and sprays may contain the usual excipients in addition to the active ingredients, e.g. lactose, talc, silica, aluminum hydroxide, calcium silicate and polyamide powder or mixtures of these substances. Sprays may additionally contain the usual propellants.

Solutions and emulsions may contain, in addition to the active ingredient(s), the usual excipients such as solvents, solubilizers and emulsifiers, e.g. Water, physiological saline solution (0.9%), ethyl alcohol, isopropyl alcohol, ethyl carbonate, ethyl acetate, benzyl alcohol, benzyl benzoate, propylene glycol, 1,3-butylene glycol, dimethylformamide, oils, especially cottonseed oil, peanut oil, corn oil, olive oil, castor oil and sesame oil, glycerol, glycerin formal, tetrahydrofurfuryl alcohol, polyethylene glycols and fatty acid esters of sorbitan or mixtures of these substances.

Suspensions may contain, in addition to the active ingredient(s), the usual excipients such as liquid diluents, e.g., water, ethyl alcohol, propylene glycol, suspending agents, e.g., ethoxylated isostearyl alcohols, polyoxyethylene sorbitol and sorbitan esters, microcrystalline cellulose, aluminum metahydroxide, bentonite, agar agar, and tragacanth, or mixtures of these substances.

The above formulation forms may also contain coloring agents, preservatives, and odor- and taste-enhancing additives, e.g., peppermint oil and eucalyptus oil, and sweeteners, e.g., saccharin.

The combination preparation according to the invention can be administered parenterally or orally. In particular, the application can be intravenous (i.v.).

1.-9. (canceled)

10. A pharmaceutical composition for use in the treatment of cerebral malaria comprising as active ingredient a compound of the general formula I 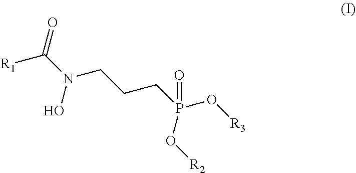
wherein R₁ is selected from the group consisting of hydrogen and methyl,
and wherein R₂ and R₃ are independently selected from the group consisting of hydrogen, substituted or unsubstituted alkyl, substituted or unsubstituted acyl, substituted or unsubstituted aryl, substituted or unsubstituted aralkyl, substituted or unsubstituted cycloalkyl, substituted or unsubstituted aryl, substituted or unsubstituted aralkyl, substituted or unsubstituted cycloalkyl, substituted or unsubstituted silyl, substituted or unsubstituted heterocyclic radical, or together form a substituted or unsubstituted C₁₋₅ alkyl chain, wherein the alkyl groups may be saturated or contain one or more double bonds or triple bonds,
in combination with a second and third pharmaceutical agents clindamycin and artesunate.

11. The pharmaceutical composition according to claim 10, wherein R₁, R₂ and R₃ are each hydrogen or formula I is fosmidomycin.

12. A medicament containing the composition according to claim 10 for use in the treatment of cerebral malaria, wherein the patient is selected from the group of children 0-14 years.

13. A medicament containing the composition according to claim 10 for use in the treatment of cerebral malaria, wherein the patient is selected from the group of children 0-10 years.

14. The pharmaceutical composition according to claim 10, wherein, in one dose there is present three times the amount (weight in mg) of formula I versus clindamycin, and/or 7.5 to fifteen times the amount (weight in mg) of formula I versus artesunate.

15. The pharmaceutical composition according to claim 10, wherein, in one dose there is present three times the amount (weight in mg) of fosmidomycin versus clindamycin, and/or 7.5 to fifteen times the amount (weight in mg) of fosmidomycin versus artesunate.

16. The pharmaceutical composition according to claim 10, wherein a dose is 1-6 mg artesunate, 5-15 mg clindamycin and 10-40 mg according to formula I per kg body weight of a patient.

17. The pharmaceutical composition according to claim 10, wherein a dose is 2-4 mg artesunate, 10 mg clindamycin and 30 mg according to fosmidomycin per kg body weight of a patient.

18. The medicament for use in the treatment of cerebral malaria according to claim 12, wherein the dose is administered to a patient within 12 hours.

19. The medicament for use in the treatment of cerebral malaria according to claim 12, wherein the dose is administered consecutively every 12 hours within 3-5 days to a patient.

20. The pharmaceutical composition according to claim 10, characterized in that the treatment is for acute therapy.

21. The medicament for use in the treatment of cerebral malaria according to claim 12, characterized in that the administration or application is parenterally or orally.

22. The medicament for use in the treatment of cerebral malaria according to claim 21, characterized in that the administration or application is intravenously.
